(19) United States
(12) Patent Application Publication
(10) Pub. No.: US 2026/0083869 A1
(43) Pub. Date: Mar. 26, 2026

(54) STERILIZATION SYSTEMS AND METHODS

(71) Applicant: Sterile Solutions International, Inc., Wellington, FL (US)

(72) Inventors: Ralph M. SIAS, Oceanside, CA (US); Bradley H. BUCHANAN, Eastsound, WA (US); Therese M. STEWART, Wellington, FL (US)

(21) Appl. No.: 19/270,000

(22) Filed: Jul. 15, 2025

Related U.S. Application Data
(63) Continuation-in-part of application No. 17/751,443, filed on May 23, 2022, now Pat. No. 12,357,717.
(60) Provisional application No. 63/207,770, filed on Mar. 22, 2021.

(51) Int. Cl. A61L 2/14

(52) U.S. Cl. CPC A61L 2/14; A61L 2/208; A61L 2103/05; A61L 2103/23; A61L 2202/11; A61L 2202/122; A61L 2202/13; A61L 2202/15

(57) ABSTRACT

A sterilization system for sterilizing an article may include a hydroxyl generation system configured to generate a first flow of hydroxyl ions and a gas transport system configured to transport the first flow of hydroxyl ions to the article and expose the article to the first flow of hydroxyl ions to sterilize the article.

### CROSS-REFERENCE TO RELATED APPLICATIONS

The present application is a continuation-in-part of U.S. patent application Ser. No. 17/751,443 filed on May 23, 2022, entitled STERILIZATION SYSTEMS AND METHODS, issued on Jul. 15, 2025 as U.S. Pat. No. 12,357,717, which claims the benefit of U.S. Provisional Application No. 63/207,770, entitled PHOTO-CATALYTIC CONTROLLED DECOMPOSITION OF H2O2 FOR DECONTAMINATION AND STERILIZATION, which was filed on Mar. 22, 2021. The foregoing are incorporated by reference herein as though set forth in their entirety.

### TECHNICAL FIELD

The present disclosure relates to systems and methods for sterilization and/or sanitation of articles. More precisely, the present disclosure relates to sterilization through the use of hydroxyl ions.

### BACKGROUND

Sterilization is a process that destroys or eliminates microbial life. It may be carried out by physical and/or chemical methods. Every day, hospitals and other health care facilities perform various surgical procedures that require invasive access to patient tissue. The medical device or the surgical instrument that contacts the patient's internal tissues may beneficially be sterilized prior to use to reduce the risk of introducing pathogens into the patient. Several outbreaks and infections have been reported in healthcare facilities, due to improper sterilization of devices used in surgical procedures. Therefore, enhanced decontamination techniques for medical and surgical devices are needed.

### SUMMARY

The various systems and methods of the present disclosure have been developed in response to the present state of the art, and in particular, in response to the problems and needs in the art that have not yet been fully solved by currently available sterilization systems and methods.

In some embodiments, a sterilization system for sterilizing an article may include a hydroxyl generation system configured to generate a first flow of hydroxyl ions and a gas transport system configured to transport the first flow of hydroxyl ions to the article and expose the article to the first flow of hydroxyl ions to sterilize the article.

In the sterilization system of any preceding paragraph, the sterilization system may further include a hydrogen peroxide generation system configured to generate hydrogen peroxide gas from a peroxide complex.

In the sterilization system of any preceding paragraph, the peroxide complex may include carbamide peroxide and one of ethanol, diethyl ether, chloroform, dichloromethane, acetone, glycerol, sodium dihydrogen pyrophosphate, aluminum oxide powder, iron oxide powder, and ceramic powder.

In the sterilization system of any preceding paragraph, the hydrogen peroxide generation system may include a first chamber configured to receive the peroxide complex and a heating element configured to heat the peroxide complex to release the hydrogen peroxide gas from the peroxide complex.

In the sterilization system of any preceding paragraph, the sterilization system may further include a hydroxyl radical generation system configured to produce a second flow of hydroxyl radicals that may cause deterioration of the hydrogen peroxide gas to produce the first flow of hydroxyl ions.

In the sterilization system of any preceding paragraph, the hydroxyl radical generation system may include a UV light source and a hydroxyl radical source, wherein the UV light source may be configured to generate a UV light that reacts with the hydroxyl radical source to produce the second flow of hydroxyl radicals.

In the sterilization system of any preceding paragraph, the hydroxyl radical source may include titanium dioxide.

In the sterilization system of any preceding paragraph, the sterilization system may further include a sterilization chamber configured to receive the first flow of hydroxyl ions and expose the article to the first flow of hydroxyl ions within the sterilization chamber, wherein the sterilization chamber may be separate from the hydroxyl generation system.

In the sterilization system of any preceding paragraph, the sterilization chamber may be configured to receive a neutralizing agent configured to neutralize residual hydroxyl radicals in the sterilization chamber.

In some embodiments, a sterilization system for sterilizing an article may include a hydroxyl generation system configured to produce hydroxyl ions from a gas and a sterilization chamber configured to expose the article to the hydroxyl ions to sterilize the article. The sterilization chamber may be separate from the hydroxyl generation system.

In the sterilization system of any preceding paragraph, the gas may include one of hydrogen peroxide and ozone.

In the sterilization system of any preceding paragraph, the sterilization system may further include a gas transport system configured to transport the hydroxyl ions to the article and expose the article to the hydroxyl ions to sterilize the article.

In the sterilization system of any preceding paragraph, the gas transport system may be further configured to urge the hydroxyl ions from the hydroxyl generation system to the article by urging a second gas under pressure through the sterilization system.

In the sterilization system of any preceding paragraph, the second gas may be non-reactive with the hydroxyl ions.

In the sterilization system of any preceding paragraph, the sterilization chamber may be configured to receive a neutralizing agent configured to neutralize residual hydroxyl radicals in the sterilization chamber.

In some embodiments, a sterilization system for sterilizing an article may include a hydrogen peroxide generation system configured to generate hydrogen peroxide and a hydroxyl ion generation system configured to produce hydroxyl ions from the hydrogen peroxide through base-catalyzed decomposition. The sterilization system may be configured to expose the article to the hydroxyl ions to sterilize the article.

In the sterilization system of any preceding paragraph, the sterilization system may further include a sterilization chamber configured to expose the article to the hydroxyl ions to sterilize the article, wherein the sterilization chamber may be separate from the hydroxyl ion generation system.

In the sterilization system of any preceding paragraph, the sterilization chamber may be configured to receive a neutralizing agent configured to neutralize residual hydroxyl radicals in the sterilization chamber.

In the sterilization system of any preceding paragraph, the hydrogen peroxide generation system may be further configured to generate the hydrogen peroxide from a peroxide complex.

In the sterilization system of any preceding paragraph, the peroxide complex may include carbamide peroxide and one of ethanol, diethyl ether, chloroform, dichloromethane, acetone, glycerol, sodium dihydrogen pyrophosphate, aluminum oxide powder, iron oxide powder, and ceramic powder.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings in which:

FIG. 1 is a flow diagram of a sterilization process according to an embodiment of the present disclosure.

FIG. 2 is a schematic view of a sterilization system that implements the sterilization process of FIG. 1, according to an embodiment of the present disclosure.

FIG. 3 is a table showing the time and results obtained with the sterilization system of FIG. 2, compared against the results obtained via application of UV light and hydrogen peroxide.

FIG. 4 is a schematic view of a sterilant filling system according to an embodiment of the present disclosure.

FIG. 5 is a schematic view of a sterilization system including a peroxide complex application system according to an embodiment of the present disclosure.

FIG. 6 is a structural formula diagram showing hydroxide ions and hydroxyl radicals.

It is to be understood that the drawings are for purposes of illustrating the concepts of the disclosure and are not to scale. Furthermore, the drawings illustrate exemplary embodiments and do not represent limitations to the scope of the disclosure.

### DETAILED DESCRIPTION

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, systems, and methods, as represented in the Figures, is not intended to limit the scope of the disclosure, as claimed, but is merely representative of exemplary embodiments of the disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

The following examples have been included to provide guidance to one of ordinary skill in the art for practicing representative embodiments of the presently disclosed subject matter. In light of the present disclosure and the general level of skill in the art, those of skill in the art can appreciate that the following examples are intended to be exemplary only and that numerous changes, modifications, and alterations can be employed without departing from the scope of the presently disclosed subject matter.

What makes hydrogen peroxide a superior sterilization agent may be its “unbalanced” molecular composition. Since hydrogen peroxide is a water molecule with an added oxygen atom, it is unstable and may seek to decompose into its stable components (water and oxygen). It essentially decomposes in the following order:

2[H₂O₂]→4[OH—]→2[H₂O]+O₂

While there may be other transition components during decomposition (H++HO₂− etc.) the ones which affect sterilization are the hydroxide ions, i.e., OH—. These are commonly referred to as hydroxyls or hydroxyl ions when they are not part of a larger molecule.

OH— ions may be different from the OH radical. The OH— ion is reactive because of its negative charge and may ionically bond to other molecules. The OH radical is charge neutral but may have an unpaired electron spin configuration that may make it very unstable. Because of its high degree of instability, the radical may be very short lived and may only be present for fractions of a second.

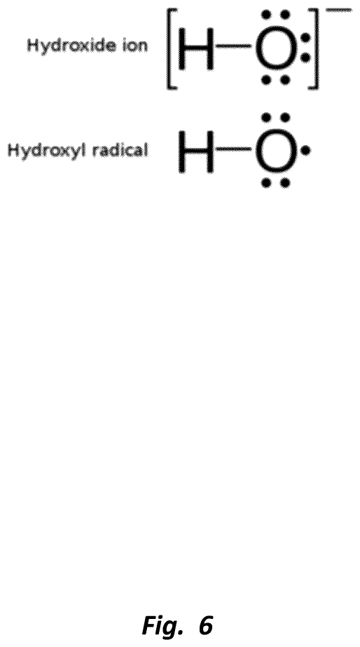

Because of their negative charge, the hydroxyl ions may create oxidation/reduction (redox) reactions with organic molecules such as microorganisms and many chemical contaminants, as shown in FIG. 6.

The redox reaction may kill microorganisms by lysing (rupturing) the cell walls. Further, this reaction may be effective for removing and/or deactivating chemical contaminants by changing their molecular structure.

The effectiveness of hydrogen peroxide for sterilization or high-level disinfection may be enhanced by controlling the rate of H₂O₂ decomposition. The rate may be such that OH— ions are produced at a sufficient quantity for a sufficient time to reach the contaminating microorganisms. Sterility may be defined as the elimination of substantially all microbial life, including bacterial spores. Elimination of “substantially all” microbial life means elimination of sufficient microbial life to avoid infection when the sterilized article is used as intended. In some examples, sterilization may relate to a process that achieves at least a 6 log 10 reduction (99.9999%) of endospores while high level decontamination may require a 6 log 10 reduction of Mycobacteria.

A Sterility Assurance Level (SAL) is a quantitative measure of the probability that an article will be non-sterile after undergoing a sterilization process. SAL is expressed as a negative exponent of 10. An SAL of 10⁻⁶ is a common target for sterilization processes, meaning there is a one in a million chance of a microorganism surviving the sterilization prosses.

The decomposition rate of hydrogen peroxide may be difficult to control due to the effects of hydrogen bonding between water and hydrogen peroxide molecules and the reactivity of the hydroxyl ions. The hydrogen bonding may keep the hydrogen peroxide bound in the aqueous solution until a reaction causes an avalanche of decomposition. Reaction rates may be very slow (no catalyst involved) or very fast (with the addition of a catalyst).

A good example of different rates of decomposition may be the reaction seen when a 3% hydrogen peroxide solution is poured onto an open wound. The hydrogen peroxide may stay bound with the water until it contacts open bonding sites, primarily catalase & glutathione in the wound. Upon contact, virtually all of the hydrogen peroxide may decompose rapidly as seen in the foaming within the wound. The decomposition may be so fast and intense that the reacting OH— ions may kill microorganisms and tissue.

There are several products currently on the market that utilize a type of hydrogen peroxide sterilization of medical instruments. Each of these products has limitations that make them less than ideal. A first product, distributed by J&J, controls the rate of H₂O₂ decomposition by pulling a high vacuum in a chamber (creating a water free environment), injecting aqueous hydrogen peroxide (30% H₂O₂), and charging the resulting vapor with high power RF (microwave) to create a plasma. The OH— ions are created quickly and maintained by the plasma until the cycle is complete. Additionally, OH radicals are present in transitional states while the plasma is present. The J&J system provides effective sterilization but is expensive and complex due to the need for the vacuum tank, pumps, RF generator, impedance matching network, etc. It is also slow because of the time required to pull the vacuum.

A second product, distributed by Steris, controls the rate by drying the air in an enclosure prior to flashing aqueous hydrogen peroxide (around 30%) off a hot surface into the dry air. There is no external energy applied to create decomposition, therefore the rate is controlled by temperature, constant water content, and the constant addition of new H₂O₂. The system is very slow because of the time required to dry the air and the highly variable density of OH— ions available during the cycle.

Another product distributed by Bioquell applies an aerosol (or fog) of 30% H₂O₂ to a sealed volume (i.e., a room or chamber). The aerosol moistens the surfaces within the volume with the H₂O₂ solution which allows the OH— ions to be released from the water when bonding sites are available. After a suitable dwell time, the moistened surfaces are neutralized and dried, and the room is vented. The Bioquell system is slow and wet and requires manpower to physically seal a room prior to treatment. Initial humidity in the room affects the length of the process which makes each cycle variable.

Each of these products has limitations as to the practicality and the effectiveness of the process. One common feature of all of these products is the use of aqueous hydrogen peroxide. Aqueous solutions of hydrogen peroxide have limitations on their use in sterilization. One of the issues with aqueous solutions, or with having water of any kind in the application, is that water condenses at a lower temperature than hydrogen peroxide. Because of this, a barrier of water is often present on the surfaces to be sterilized. To reach the microorganisms and chemical contaminants, the hydrogen peroxide must diffuse through the water condensed on the surface to deactivate or destroy the contaminants. Diffusion is a slow process. Additionally, chemical reactions occur between the water and the hydrogen peroxide, limiting the amount of hydrogen peroxide available for sterilization.

The present disclosure provides many benefits over these known methods. In some embodiments, a peroxide complex, rather than an aqueous solution, may be used to generate hydrogen peroxide, avoiding the creation of a water barrier on the surfaces to be sterilized. Relatively simple and/or inexpensive components may be used to generate hydroxyl ions that can be used for rapid sterilization.

The sterilization system 200 may be configured to be used in a surgery setting to sterilize allograft and/or autograft tissue to prevent osteonecrosis. Additionally, or alternatively, the sterilization system 200 may be configured to sterilize a HVAC system including air ducts. Additionally, or alternatively, the sterilization system 200 may be configured to sterilize an Interior of foil or aluminum packaging for food. Additionally, or alternatively, the sterilization system 200 may be configured so that a sterilization chamber 205 may be sized and/or configured based on a size, a geometry, and/or a material of an article 204 to be sterilized.

Additionally, or alternatively, the sterilization system 200 may be configured to be powered by AC voltage and/or DC voltage. Additionally, or alternatively, the sterilization system 200 may be configured as an air decontamination device. Additionally, or alternatively, the sterilization system 200 may be configured to sterilize lumens/waterlines (possible male part to insert in lumen of waterlines).

Additionally, or alternatively, the sterilization system 200 may be configured to sterilize a prefilled syringe. Additionally, or alternatively, the sterilization system 200 may be scalable, and each component of the sterilization system 200 may be scalable, based on a desired size, quantity, geometry, and/or material to be sterilized.

In an embodiment, the sterilization system 200 may include an electronic control unit 245 configured to control operating parameters of the sterilization system 200. The electronic control unit 245 may include a user interface, a data storage unit, and/or an input/output interface (for example a USB port). The electronic control unit 245 may be electrically coupled to the heating element 206 and/or the UV light source 243. Additionally, or alternatively, the electronic control unit 245 may be electrically coupled to a pressure sensor and/or a pressure regulator.

In an embodiment, the sterilization system 200 may be sized and configured for consumer use, for example to sterilize and/or decontaminate eye glasses, mouth guards, cell phones, ear pods, and/or other consumer items.

As described above, the hydroxyl ions may be the primary sterilization agent in the sterilization system 200. The sterilization system 200 may include a hydroxyl generation system configured to generate hydroxyl radicals and/or hydroxyl ions from a gas such as ozone and/or hydrogen peroxide gas.

In an embodiment, ozone may be used in place of, or in addition to, hydrogen peroxide gas as a source for creating hydroxyl ions. Through a cascade of radical reactions, ozone in aqueous environments—especially under alkaline or catalytic conditions—may ultimately produces hydroxyl radicals and/or hydroxyl ions.

Under alkaline conditions, ozone may react with hydroxide ions naturally present in water. The mechanism for hydroxyl radical formation from ozone may generally proceed via a multi-step reaction sequence, initiated by the reaction of ozone with hydroxide ions (OH⁻):

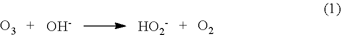

The hydroperoxide anion (HO₂⁻) formed in Equation (1) may exist in equilibrium with the superoxide radical (O₂·⁻):

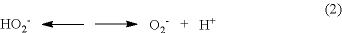

The superoxide radical may subsequently react with another molecule of ozone to produce the ozonide radical (O₃·⁻):

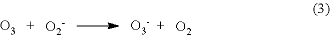

Finally, the ozonide radical may react with water to yield a hydroxyl radical, a hydroxide ion, and molecular oxygen:

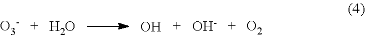

Collectively, this reaction sequence may provide a pathway for the in-situ generation of hydroxyl radicals from ozone in aqueous systems. These radicals may be capable of initiating rapid and non-selective oxidation of a wide variety of organic and inorganic contaminants. The efficiency of this process may be further enhanced through the use of catalysts, which may promote the decomposition of ozone or intermediate radicals, thereby increasing the local concentration of hydroxyl radicals and/or hydroxyl ions and improving overall contaminant degradation and/or sterilization efficiency.

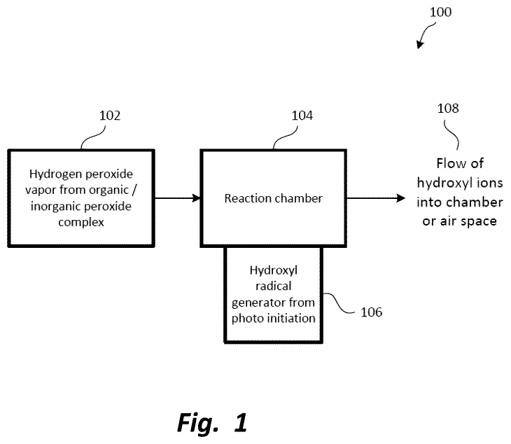

FIG. 1 is a flow diagram of a sterilization process 100 according to one embodiment. Hydrogen peroxide gas may be produced through the use of a peroxide complex 102. The gaseous hydrogen peroxide may be transported into a reaction chamber 104. In the reaction chamber 104, hydroxyl radicals produced by a hydroxyl radical generator 106 may react with the hydrogen peroxide to produce a flow of hydroxyl ions 108. The hydroxyl ions may be produced in the hydroxyl radical generator 106 by photo initiation. Once the hydrogen peroxide has been decomposed by the hydroxyl radicals into the flow of hydroxyl ions 108, the hydroxyl ions 108 may be transported to a sterilization chamber or other sterilization area, at which the hydroxyl ions 108 may react with microorganisms and chemical contaminants to deactivate and/or destroy them as described above.

In an embodiment, the sterilization system 200 may be configured to produce hydroxyl ions from hydrogen peroxide without the use of hydroxyl radicals. Hydrogen peroxide (H₂O₂) may be used to generate hydroxyl ions (OH⁻) through non-radical mechanisms, avoiding the formation of reactive hydroxyl radicals (·OH). This may be particularly advantageous in applications where controlled pH modulation is desired without inducing oxidative degradation via free radical pathways. The sterilization system 200 may include a hydroxyl ion generation system configured to produce hydroxyl ions from hydrogen peroxide through base-catalyzed decomposition.

Under alkaline conditions, hydrogen peroxide may undergo base-catalyzed decomposition that results in the formation of hydroxyl ions and molecular oxygen. The reaction may proceed via the following steps:

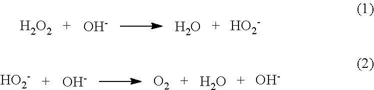

These reactions may occur without the formation of hydroxyl radicals and result in the net generation or maintenance of hydroxyl ions in solution. This mechanism may be particularly effective in buffered or strongly alkaline aqueous environments.

Hydrogen peroxide may also undergo decomposition through non-radical disproportionation in the presence of heterogeneous catalysts such as platinum (Pt), manganese dioxide (MnO₂), or other non-redox active surfaces. The catalytic reaction may proceed as follows:

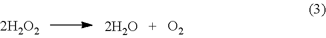

This pathway may avoid the generation of hydroxyl radicals and can contribute to a rise in solution pH depending on the system's buffering capacity and overall chemical environment.

These non-radical pathways may allow for the controlled release or generation of hydroxyl ions from hydrogen peroxide, enabling pH adjustment or enhanced reactivity without initiating free radical oxidation. Such approaches may be useful in chemical treatment systems, controlled oxidation reactions, and processes requiring precise control over oxidative species.

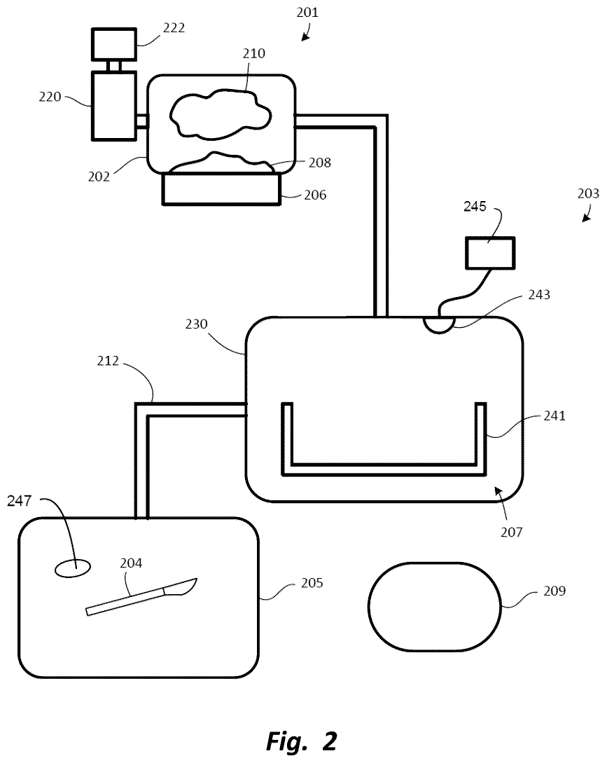

FIG. 2 is a schematic diagram of a sterilization system 200 according to one embodiment. The sterilization system 200 may include a hydrogen peroxide generation system 201, a reaction system 203, and a sterilization chamber 205 containing an article 204 to be sterilized.

The hydrogen peroxide generation system 201 may include a chamber 202 and a heating element 206. A peroxide complex 208 may be placed in the chamber. The peroxide complex may optionally be placed directly on the heating element 206, and/or on an interior wall of the chamber 202 adjacent to the heating element 206. Peroxide compounds are chemical compounds that have hydrogen peroxide bonded to another compound. The hydrogen peroxide can be dissociated from the other compound by heat and/or chemical reactions. The peroxide complex may be organic or inorganic. Some examples of peroxide complexes include carbamide peroxide, also known as urea peroxide. Carbamide peroxide has a chemical composition of CH₆N₂O₃ and a molecular weight of 94.07 g/mol. Hydrogen peroxide has a chemical composition of H₂O₂ and a molecular weight of 34.0147 g/mol. As the carbamide peroxide decomposes, 36.1% of the weight of the carbamide is hydrogen peroxide. To create 1 mol of hydrogen peroxide requires 94.59 g of carbamide peroxide. Said another way, the ratio of hydrogen peroxide in carbamide peroxide is 1:3, or the hydrogen peroxide is one third the weight of the carbamide peroxide.

Advantageously, the peroxide complex 208 may be stabilized for extended storage through addition of a stabilizing agent. The stabilizing agent may include chelating agents (EDTA) which may be suitable if ferric compounds are not used to chemically aid H₂O₂ generation within the sterilization system 200.

Additionally, or alternatively, phosphates (sodium dihydrogen pyrophosphate) may be used to stabilize pH of the peroxide complex 208, which may help to prevent decomposition. Phosphates may be generally acceptable for the process in lower quantities. Higher quantities of phosphates may hinder the release of H₂O₂ from the peroxide complex 208 with the sterilization system 200.

Additionally, or alternatively, silica and/or other moisture absorbent materials may bind water molecules, thereby preventing hydrolysis, which may cause dissociation. The silica and/or other moisture absorbent materials may be advantageous within the sterilization system 200 because they may have good temperature transmission characteristics.

Additionally, or alternatively, a compound including a peroxide complex 208 glycerol solution may be stabilized with succinic anhydride (at approximately 0.02%). The peroxide complex 208 glycerol solution may have a boiling point around 290° C., well above the 50° C. to 75° C. of the sterilization system 200 process to outgas the H₂O₂.

Additions to the any of the above powders and/or pastes to aid in generation of dry H₂O₂ (without adding water) may include: Ethanol (EtOH), Diethyl ether (Et₂O), Chloroform (CHCl₃), Dichloromethane (CH₂CL₂), Acetone (Me₂CO), and/or Glycerol.

The heating element 206 may include a heating coil, and/or another resistive heating material, coupled with an electric power source. The heating coil may be configured to convert electric current to heat. The amount of heat generated by the coil may be proportional to the electric current provided by the electric power source and flowing through the coil. The heating element 206 may be configured to regulate a temperature within the chamber 202 and thereby heating the peroxide complex 208 through convection. Additionally, or alternatively, the heating element 206 may be configured to heat the peroxide complex 208 directly through conduction.

Additionally, or alternatively, the heating element 206 may include an inductive heating coil configured to generate an inductive current in an electrically conductive material received within the inductive heating coil. The amount of heat generated by the inductive heating coil may be proportional to the electric current provided by the electric power source and flowing through the inductive heating coil.

The peroxide complex 208 may be configured to be heated in the chamber 202 by the heating element 206. This may release hydrogen peroxide gas 210 from the peroxide complex 208. The hydrogen peroxide generation system 201 and the reaction system 203 may be configured so that the hydrogen peroxide gas 210 may be transported to the reaction system 203. The heating element 206 may be configured to regulate the temperature of the chamber 202 and/or the peroxide complex 208 to provide the optimal flow rate of hydrogen peroxide from the hydrogen peroxide generation system 201 to the reaction system 203. In some embodiments, the heating element 206 may be configured to heat the peroxide complex 208 by means of conduction and/or convection to a temperature within the range of 0° C. to 100° C. More precisely, the heating element 206 may be configured to heat the peroxide complex 208 by means of conduction and/or convection to a temperature within the range of 30° C. to 80° C. Yet more precisely, heating element 206 may be configured to heat the peroxide complex 208 by means of conduction and/or convection to a temperature within the range of 50° C. to 70° C. Still more precisely, the heating element 206 may be configured to heat the peroxide complex 208 by means of conduction and/or convection to a temperature within the range of 55° C. to 65° C. Even more precisely, the heating element 206 may be configured to heat the peroxide complex 208 by means of conduction and/or convection to a temperature of about 60° C.

In an embodiment, the hydrogen peroxide generation system 201 may be located within the reaction chamber 230. Additionally, or alternatively, the sterilization chamber 205 may be located within the reaction chamber 230. Additionally, or alternatively, the hydrogen peroxide generation system 201 and the sterilization chamber 205 may be located in the reaction chamber 230.

In an embodiment, the sterilization system 200 may include a system chamber within which hydrogen peroxide may be generated (as described for the hydrogen peroxide generation system 201), the hydrogen peroxide may be converted to hydroxyl radicals and/or hydroxyl ions (as described for the hydroxyl radical generation system 207), and an article 204 may be sterilized by the hydroxyl radicals and/or hydroxyl ions (as described for the sterilization chamber 205).

In some embodiments, a thermostat (not shown) within the chamber 202 may be used to control the electric current provided by the power source to the heating element 206, thereby controlling the heat emitted by the heating element 206, to keep the peroxide complex 208 within the desired temperature range. Further, in some embodiments, the heating element 206 may be used to control the rate of hydrogen peroxide generation, for example, by heating the peroxide complex 208 to a higher temperature to increase the rate of hydrogen peroxide generation, or allowing reduction in the temperature of the peroxide complex 208 to slow the rate of hydrogen peroxide generation.

In some embodiments, the hydrogen peroxide gas may be transported by a gas transport system 220. In some embodiments, the gas transport system 220 may utilize ambient air to move gases throughout the system. The gas transport system 220 may be configured, through increases and decreases in pressure, to draw ambient air into the gas transport system 220 and urge the ambient air into the chamber 202 under pressure to displace the hydrogen peroxide gas 210 out of the chamber 202 and into the reaction system 203. Ambient air may be acceptable as a transport gas. However, ambient air may have a limitation in that oxygen may be a part of ambient air and may react with the hydrogen peroxide gas 210 and thereby may limit the amount of hydrogen peroxide gas 210 available in the reaction system 203. Additionally, ambient air often contains water vapor. As explained above, water vapor may be highly reactive with hydrogen peroxide and may limit the amount of hydrogen peroxide gas 210 available as well as limiting the reaction surface of the hydrogen peroxide.

One of the reasons that the inventors have developed an anhydrous hydrogen peroxide sterilization system may be that hydrogen peroxide in solution in water tends to react much more slowly. Even with gaseous hydrogen peroxide and water, the water may condense first, and may form a barrier on the surfaces which may be being sterilized, as described above. The hydrogen peroxide may still reach the surface, but it must diffuse through the water, slowing the reaction and reducing the available amount of hydrogen peroxide. Therefore, an ambient air gas transport system is usable in some conditions but can be a limiting factor in the effectiveness of the sterilization system 200.

In some embodiments, the gas transport system 220 may be configured to utilize a sealed gas in place of ambient air to move gases throughout the system. The sealed gas may be housed in a cannister 222 that may be attached to the gas transport system 220. Utilizing a gas that is non-reactive with hydrogen peroxide and with reactive oxygen species may be advantageous. Non-reactive gases include but are not limited to nitrogen and argon. The non-reactive gas may be pressurized by the gas transport system 220 and may be used to move the hydrogen peroxide gas 210 to the reaction system 203. The non-reactive gases may optionally be recaptured from the reaction system 203 and reused by the gas transport system 220.

In some embodiments, the non-reactive gas may be pure nitrogen. In alternative embodiments, the non-reactive gas may include other trace elements, for example, at a concentration of less than 20%, less than 15%, less than 10%, or less than 5% of the volume of the gas.

The reaction system 203 may include a reaction chamber 230. A hydroxyl radical generation system 207 may also be connected to the reaction chamber 230. In some embodiments, the hydroxyl radical generation system 207 may be housed within the reaction chamber 230. The hydroxyl radical generation system 207 may include a UV light source 243 and a hydroxyl radical source. The hydroxyl radical source may be configured to function as a photo initiator. UV light from the UV light source 243 may react with the photo initiator to produce hydroxyl radicals.

In an embodiment, the hydroxyl radical generation system 207 may be located within the sterilization chamber 205. The sterilization chamber 205 may further be configured to expose the article 204 to the UV light generated by the UV light source 243, in addition to the hydroxyl ions and/or hydroxyl radicals, to sterilize the article 204.

Additionally, or alternatively, the hydroxyl radical source may be configured to function through Fenton Reactions which may involve a reaction between ferrous iron and hydrogen peroxide, where Fe²⁺ acts as a catalyst to decompose H₂O₂ and produce hydroxyl radicals.

Additionally, or alternatively, the hydroxyl radical source may be configured to function through Catalytic Advanced Oxidation. Catalytic Advanced Oxidation may produce hydroxyl radicals through chemical reactions between an oxidant (commonly hydrogen peroxide or ozone) and a solid or dissolved catalyst, often a transition metal like iron, copper, or titanium.

Photo initiators include, but are not limited to, transition metal oxides, which are compounds composed of oxygen atoms bound to transition metals. Transition metal oxides are commonly used for their catalytic properties. The versatility of these molecules also leads to their used as pigments in paints and plastics. Transition metal oxides include; titanium dioxide (TiO2), strontium dioxide (SrO2), Zirconium dioxide (ZrO2), Zinc oxide (ZnO), Copper oxide (CuO), and others. Metal ions such as Copper (Cu+) are also used as photo initiators. Combinations of metal oxides with each other or with other compounds can also be strong photo initiators. Titanium dioxide is one of the most used photo initiators. It is generally safe to work with, is readily available, and is an efficient photo initiator.

Thus, in FIG. 2, the photo initiator 241 may take the form of a plate composed of titanium dioxide. The plate may be shaped to provide an extended surface area that receives the UV light from the UV light source 243, thus, enhancing the rate of hydroxyl radical production. In alternative embodiments, the photo initiator 241 may be a screen to which titanium dioxide is attached.

The titanium dioxide can be attached to the screen in a variety of ways. The titanium dioxide may be a coating applied to a substrate, such as paint or powder coating. The substrate may be metal, such as copper, iron, zinc, stainless steel, or other metals. Alternatively, the substrate may be a polymer such as plastic. The titanium dioxide may be manufactured into the screen itself such as by being an ingredient in a polymer used to form the screen. In embodiments in which the titanium dioxide is an ingredient in the polymer used to form a screen, the screen may be produced by any polymer fabrication method, including but not limited to extrusion, molding, or by an additive manufacturing method such as 3D printing.

UV light in the wavelengths of 250 nm to 450 nm are known to be effective for photo initiation. UV light in the wavelengths of 100 nm to 280 nm, otherwise known as UVC, are known to be effective for deactivating and destroying microorganisms such as viruses and bacteria. For use in a sterilization system, a UV light source that produces UV light in a wavelength of between 250 nm and 280 nm may be most beneficial for both photo initiation and deactivating and destroying microorganisms. The UV light may activate the titanium dioxide and may have the added benefit of inactivating viruses and bacteria. There are many UV light sources which produce UV light with wavelengths within the range from 250 nm to 280 nm, any of which would be effective for use in the sterilization system 200. In one embodiment, the system uses a UV light source with wavelength of 254 nm.

Additionally, or alternatively, water vapor may be added to the hydrogen peroxide generation system 201 to enhance H₂O₂ generation. The residual water may be mostly converted to H₂O₂ during photo initiation and, therefore, may not affect the “dryness” of the H₂O₂ gas.

Additionally, or alternatively, a metallic oxide powder may be added to the hydrogen peroxide generation system 201 to allow more efficient induction heating of the peroxide complex 208 through the use of an induction coil. More specifically, aluminum oxide powder and/or iron oxide powder may be added to the peroxide complex 208. The heating element 206 may then include an induction heating coil. The induction heating coil may be configured to heat the aluminum oxide powder and/or iron oxide powder, thereby heating the peroxide complex 208.

Additionally, or alternatively, a powder may be added to the peroxide complex 208 to increase heating efficiency of the peroxide complex 208 in the hydrogen peroxide generation system 201. The powder may include aluminum oxide powder, iron oxide powder, and/or ceramic powder.

The reaction chamber 230 may be configured to facilitate mixture of the hydroxyl radicals with the hydrogen peroxide gas 210 to produce hydroxyl ions. The reaction of hydroxyl ions with hydrogen peroxide gas may be a cascading reaction—as the hydrogen peroxide and hydroxyl radicals decompose to hydroxyl ions, the reaction may tend to avalanche. The rate at which the hydroxyl radicals come in contact with the hydrogen peroxide gas 210 may determine the amount of the hydroxyl ions that is produced. The amount of hydroxyl radicals may be dependent on the intensity, brightness, illuminance, and/or wavelength of UV light bombarding the photo initiator 241 to release the hydroxyl radicals. The flow of radicals can be increased by turning the UV light source 243 on and/or increasing its intensity, brightness, and/or luminance. Similarly, the flow of radicals can be reduced by turning the UV light source 243 off and/or reducing its intensity, brightness, and/or illuminance. Additionally, or alternatively, the wavelength of UV light emitted by the UV light source 243 may be adjusted to control the reaction. Additionally, in some embodiments, there may be multiple UV light sources. By using multiple UV light sources, differing rates of hydroxyl ion production can be maintained through any of the techniques mentioned above, applied to one or multiple of the UV lights sources. The rate of continuous production of hydroxyl ions from the interaction between hydrogen peroxide gas and hydroxyl radicals can be regulated from the amount, intensity, brightness, illuminance, and/or wavelength of UV light bombarding the titanium dioxide photo initiator.

In some embodiments, the product produced by the hydrogen peroxide generation system and the interaction of the hydrogen peroxide gas with the hydroxyl ions in the hydroxyl radical generation system may be predominantly hydroxyl ions; however, hydrogen peroxide and hydroxyl radicals may still be present, as the entire volume of the hydrogen peroxide gas may not decompose to hydroxyl ions.

In addition to being reactive with compounds in ambient air, the components of the sterilization product may be reactive with the material of which the generation system, reaction system, and sterilization chamber are composed. These materials may include some metals, such as copper, magnesium, and brass as well as some polymers, including acetal, nitrile, rubber, various synthetic rubbers, and other polymers. Materials that may be non-reactive with the sterilization components may include stainless steel, aluminum, polycarbonate, and polytetrafluoroethylene (PTFE). Therefore, careful design may be required to create the apparatus necessary to generate the reaction and sterilization products.

The sterilization system 200 may be configured so that, once hydroxyl ions have been produced, they may be conveyed to the sterilization chamber 205. In embodiments where the sterilization chamber 205 and the reaction chamber 230 are not the same chamber, the sterilization system 200 may be configured so that, the hydroxyl ions may be conveyed from the reaction chamber 230 to the sterilization chamber 205 through a conduit, such as conduit 212. A transport system may be configured to urge the hydroxyl ions from the reaction chamber 230 to the sterilization chamber 205, for example, via the ambient air and/or non-reactive gas that may be pressurized by the gas transport system 220. In the alternative, a different conveyance mechanism may be used to convey the hydroxyl ions to the sterilization chamber 205. For example, a different gas transport system (not shown) may be used. The conduit 212 may be of any length necessary to connect the reaction chamber 230 to the sterilization chamber 205. In some embodiments, the reaction chamber 230 and the sterilization chamber 205 may be located close to one another, such as in the same room. In these embodiments, the conduit 212 may be short due to the physical proximity of the reaction chamber 230 and the sterilization chamber 205.

In some embodiments, the sterilization chamber 205 may be in a location that may be remote from the location of the remainder of the sterilization system 200, including the hydrogen peroxide generation system 201 and the reaction system 203. In some embodiments, the sterilization system may be configured so that the hydroxyl ions may be generated at an ion production location, and then transported to a separate location (such as a patient care room), and there introduced into the sterilization chamber 205 to sterilize the article 204. Examples of this may include having a generation system and a reaction system in a designated location within a patient care facility, such as a surgery ward, and having sterilization chambers at strategic locations throughout the patient care facility, such as in each surgery room. Another example could be a dentist office, at which the generation and reaction systems are centrally located and the sterilization chambers are in each of the examination rooms. In such cases, the conduit 212 used to transport the hydroxyl ions from the reaction system 203 to the sterilization chamber 205 may be sufficiently long to reach from the reaction chamber 230 to the sterilization chamber 205.

In an embodiment, the sterilization system 200 may include a hydrogen peroxide generation system 201 and a reaction system 203 but may lack a sterilization chamber 205. The hydrogen peroxide generation system 201 may be configured to generate hydrogen peroxide, and the reaction system 203 may be configured to produce a first flow of hydroxyl radicals that may cause deterioration of the hydrogen peroxide to produce a second flow of hydroxyl ions that may be directed at an article 204 that may, or may not, be contained within a chamber. The second flow of hydroxyl ions may sterilize and/or decontaminate the article 204 upon contact of the hydroxyl ions with the article 204.

In an embodiment, the sterilization system 200 may include additional activation via gas exposure in the sterilization chamber. The gas may contain very small amounts of one of the following chemicals:
manganese dioxide (MnO₂) nano particles
potassium iodide (KI) aqueous vapor
iron iii-ferric chloride aqueous vapor
iron ii-FeSO₄ (Fenton's reagent) aqueous vapor
chloride (FeCl₃) aqueous vapor
catalase aqueous vapor

Additionally, or alternatively, the sterilization system 200 may be configured to receive a neutralizing agent 247, such as sodium thiosulfate (Na₂S₂O₃), aqueous solution as an aerosol in the sterilization chamber 205 to neutralize residual hydrogen peroxide and/or residual hydroxyl radicals in the sterilization chamber 205 after the sterilization cycle is complete.

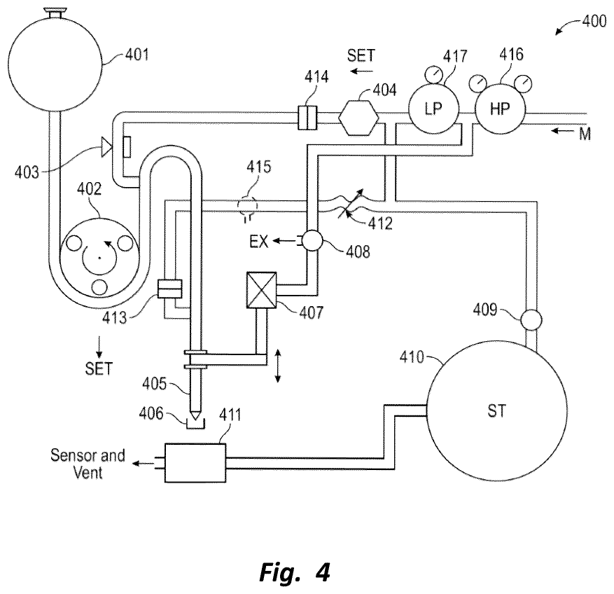

In some embodiments, the hydroxyl ions may be used to sterilize apparatus used to fill vessels, such as vessel 209, used for transporting biological substances such as growth media. The sanitization system may also sterilize at least a portion of the vessel 209 itself. These vessels may be transported to a location removed from the hydrogen peroxide generation system 201 and the hydroxyl radical generation system (the reaction system 203 of FIG. 2). An exemplary filling system, or filling system 400, is shown and described in connection with FIG. 4.

In other embodiments, the sterilization system 200 may be configured to sterilize an article directly in the reaction chamber 230. Thus, the sterilization chamber 205 of FIG. 2 may be omitted, and the article 204 may be placed directly in the reaction chamber 230 so that it will be sterilized by the hydroxyl ions produced within the reaction chamber 230. The reaction chamber 230 may be configured to enable the article 204 to be bombarded by the UV light produced by the UV light source 243, in addition to the effect of the hydroxyl ions generated within the reaction chamber 230. Where the sterilization chamber 205 is present and separate from the reaction chamber 230, the UV light produced by the UV light source 243 may still help to eliminate any microbes present in the hydroxyl ions and/or conveying gas.

The sterilization system 200 may have several advantages for deactivating or destroying contaminants. While the hydroxyl ions may be the predominant sterilizing agent, each of the components in the sterilization system 200 may be capable of deactivating and destroying contaminants. Hydroxyl radicals, hydrogen peroxide, UV light, and/or titanium dioxide may be used in other sterilization processes, and may help to sterilize the article 204 independently of the use of the hydroxyl ions. The sterilization system 200 may be relatively simple, and the various components that make up the sterilization system 200 may be relatively inexpensive and easy to fabricate or obtain. Use of anhydrous hydrogen peroxide may expedite sterilization of the article 204 and/or reduce the amount of material (for example, the peroxide complex 208 and/or the photo initiator 241). Sterilization may be quick and reliable. The ability to locate the sterilization chamber 205 remote from the remainder of the sterilization system 200 may beneficially facilitate performance of local sterilization, and may avoid and/or reduce the need to ship instruments to specialized facilities for sterilization.

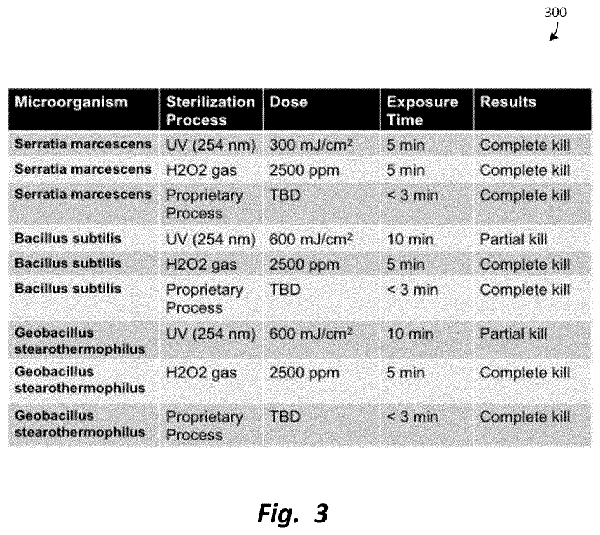

The sterilization system 200 has been tested using biological indicators to represent contaminating microorganisms. Tests were run comparing the effectiveness of UV light sterilization compared to the use of hydrogen peroxide gas and then to the process of using the sterilization system 200. UV light in the 254 nm wavelength was used. There is evidence of the effectiveness of the 254 nm wavelength being an effective sterilization tool at least against certain bacteria and viruses. The hydrogen peroxide gas was present in a vaporized aqueous solution. There were three different strains of bacterial contaminants. The results are shown in a table 300 in FIG. 3.

As can be seen in the table 300, the use of the sterilization system 200 was the most effective and the quickest. UV light alone does not kill all of the bacterial strains. The hydrogen peroxide gas killed all the bacteria and did so in about 5 minutes. The use of the sterilization system 200, including production of anhydrous hydrogen peroxide with hydroxyl radicals to produce hydroxyl ions, was completely effective and took the least amount of time.

In some embodiments, the hydroxyl ions may be used to sterilize a system for transporting biological substances, including the filling apparatus and at least a portion of the vessels used for transport. A sterilization system such as that described previously may be used to supply the hydroxyl ions. An exemplary filling system, or filling system 400, is shown and described in connection with FIG. 4.

Referring to FIG. 4, the filling system 400 may include a fluid media filter 401, a roller pump 402, a pinch valve 403, a sterilizing nitrogen filter 404, a fill tube 405, a fill tube cap 406, an actuator 407, a five-way spool valve 408, a solenoid valve 409, a sterilizing gas generator 410, a fill valve body 411, a flow restrictor valve 412, a CPC Sterile Connecter 413, a CPC Sterile connecter 414, a solenoid valve 415, a high-pressure nitrogen regulator 416, and a low-pressure nitrogen regulator 417.

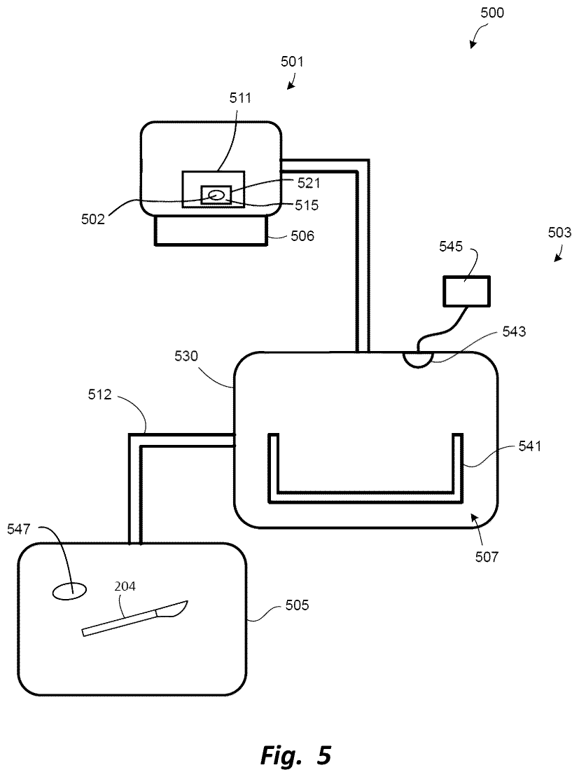

FIG. 5 is a schematic view of a sterilization system 500 including a peroxide complex application system 511 according to an embodiment of the present disclosure. The various components of the sterilization system 500 with the corresponding reference numbers as those of the sterilization system 200 may be similar in function and/or configuration to their counterparts of the sterilization system 200. Differences between the sterilization system 200 and the sterilization system 500 will be set forth below.

The sterilization system 500 may include a hydrogen peroxide generation system 501, a reaction system 503, a sterilization chamber 505, an electronic control unit 545, and a conduit 512 connecting the reaction system 503 and the sterilization chamber 505. The reaction system 503 may include a reaction chamber 530, a hydroxyl radical generation system 507 including a UV light source 543 and a photo initiator 541. The sterilization chamber 505 may be configured to receive a neutralizing agent 547.

The hydrogen peroxide generation system 501 may include a peroxide complex application system 511 and a heating element 506. The peroxide complex application system 511 may include a peroxide complex packet 515, such as a sealed bag, a sachet, a cup, a tray and/or other packaging configuration that may contain the appropriate quantity of peroxide complex 502 to achieve a sterilization process in the sterilization system 500. The peroxide complex application system 511 may include metal, ceramic or plastic material that is compatible with the peroxide complex 502 and the outgassing at a reaction temperature of the sterilization process.

The peroxide complex application system 511 may have one or more sides 521 having a porous material that may allow outgassing at the reaction temperature. One such material may be DuPont's Tyvek. The peroxide complex application system 511 may be configured such that the remains of the peroxide complex 502, after the sterilization process is completed, may be easily removed in preparation for a subsequent sterilization process of the sterilization system 500.

The sterilization process may be used for sterilizing the filling apparatus which is used to fill a biological transport container such as the vessel 209 of FIG. 2. The filling sequence may proceed as follows:
1. Prepare the system by getting the load set;
2. Turn on the transport system, which may use a sealed nitrogen tank to supply the transport gas;
3. Remove the protective shipping cover from the fill valve so that the fill valve can connect to the container;
4. Begin the production of the sterilization compounds by heating the Carbamide Peroxide in the reaction chamber;
5. Load the bag fill port to the port lifter;
6. Raise the fill port;
7. Initiate the production of sterilizing gas by activating the UV light source and bombarding the photo initiator, titanium dioxide, with UV light to produce hydroxyl radicals, and transport the hydrogen peroxide gas to the reaction chamber;
8. Open the sterilization valve and open the exhaust valve to induce flow;
9. Close the exhaust valve is closed;
10. Initiate a timed sterilant exposure to produce the correct amount of sterilant;
11. Close the sterilization valve;
12. Open the exhaust valve again;
13. Open the nitrogen flush valve;
14. Close the exhaust and nitrogen valve;
15. Lower the fill valve to pierce the bag port top membrane and connect the fill tube to the empty bag;
16. Fill to the desired volume by cycling the roller pump;
17. Once the bag is filled, use a short burst of nitrogen to clear the bag port and tubing of liquid to eliminating dripping;
18. Seal the bag fill tube closed; and
19. Retract the filler fill tube.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. As defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, and methods disclosed herein.

1. A sterilization system for sterilizing an article, the sterilization system comprising:
a hydroxyl generation system configured to generate a first flow of hydroxyl ions; and
a gas transport system configured to transport the first flow of hydroxyl ions to the article and expose the article to the first flow of hydroxyl ions to sterilize the article.

2. The sterilization system of claim 1, further comprising a hydrogen peroxide generation system configured to generate hydrogen peroxide gas from a peroxide complex.

3. The sterilization system of claim 2, wherein the peroxide complex comprises carbamide peroxide and one of ethanol, diethyl ether, chloroform, dichloromethane, acetone, glycerol, sodium dihydrogen pyrophosphate, aluminum oxide powder, iron oxide powder, and ceramic powder.

4. The sterilization system of claim 2, wherein the hydrogen peroxide generation system comprises a first chamber configured to receive the peroxide complex and a heating element configured to heat the peroxide complex to release the hydrogen peroxide gas from the peroxide complex.

5. The sterilization system of claim 2, further comprising a hydroxyl radical generation system configured to produce a second flow of hydroxyl radicals that causes deterioration of the hydrogen peroxide gas to produce the first flow of hydroxyl ions.

6. The sterilization system of claim 5, wherein the hydroxyl radical generation system comprises a UV light source and a hydroxyl radical source, wherein the UV light source is configured to generate a UV light that reacts with the hydroxyl radical source to produce the second flow of hydroxyl radicals.

7. The sterilization system of claim 6, wherein the hydroxyl radical source comprises titanium dioxide.

8. The sterilization system of claim 1, further comprising a sterilization chamber configured to receive the first flow of hydroxyl ions and expose the article to the first flow of hydroxyl ions within the sterilization chamber, wherein the sterilization chamber is separate from the hydroxyl generation system.

9. The sterilization system of claim 8, wherein the sterilization chamber is configured to receive a neutralizing agent configured to neutralize residual hydroxyl radicals in the sterilization chamber.

10. A sterilization system for sterilizing an article, the sterilization system comprising:
a hydroxyl generation system configured to produce hydroxyl ions from a gas; and
a sterilization chamber configured to expose the article to the hydroxyl ions to sterilize the article;
wherein the sterilization chamber is separate from the hydroxyl generation system.

11. The sterilization system of claim 10, wherein the gas comprises one of hydrogen peroxide and ozone.

12. The sterilization system of claim 10, further comprising a gas transport system configured to transport the hydroxyl ions to the article and expose the article to the hydroxyl ions to sterilize the article.

13. The sterilization system of claim 12, wherein the gas transport system is further configured to urge the hydroxyl ions from the hydroxyl generation system to the article by urging a second gas under pressure through the sterilization system.

14. The sterilization system of claim 13, wherein the second gas is non-reactive with the hydroxyl ions.

15. The sterilization system of claim 10, wherein the sterilization chamber is configured to receive a neutralizing agent configured to neutralize residual hydroxyl radicals in the sterilization chamber.

16. A sterilization system for sterilizing an article, the sterilization system comprising:
a hydrogen peroxide generation system configured to generate hydrogen peroxide; and
a hydroxyl ion generation system configured to produce hydroxyl ions from the hydrogen peroxide through base-catalyzed decomposition;
wherein the sterilization system is configured to expose the article to the hydroxyl ions to sterilize the article.

17. The sterilization system of claim 16, further comprising a sterilization chamber configured to expose the article to the hydroxyl ions to sterilize the article, wherein the sterilization chamber is separate from the hydroxyl ion generation system.

18. The sterilization system of claim 17, wherein the sterilization chamber is configured to receive a neutralizing agent configured to neutralize residual hydroxyl radicals in the sterilization chamber.

19. The sterilization system of claim 16, wherein the hydrogen peroxide generation system is further configured to generate the hydrogen peroxide from a peroxide complex.

20. The sterilization system of claim 19, wherein the peroxide complex comprises carbamide peroxide and one of ethanol, diethyl ether, chloroform, dichloromethane, acetone, glycerol, sodium dihydrogen pyrophosphate, aluminum oxide powder, iron oxide powder, and ceramic powder.
